(19) United States
(12) Patent Application Publication
(10) Pub. No.: US 2001/0003751 A1
(43) Pub. Date: Jun. 14, 2001

(54) Pharmaceutical composition for treating transient ischemic attack

(76) Inventors: Zen-ichi Terashita, Osaka (JP); Kaneyoshi Kato, Hyogo (JP); Takenobu Sohma, Tokyo (JP)

Correspondence Address: Harold C. Wegner, FOLEY & LARDNER, Washington Harbour, 3000 K Street, N.W., Suite 500, Washington, DC, 20007-5109, US

(21) Appl. No.: 09/769,440

(22) Filed: Jan. 26, 2001

Related U.S. Application Data
(63) Continuation of application No. 08/669,316, filed on Jul. 9, 1996, now abandoned.
Application No. 08/669,316 is a 371 of international application No. PCT/JP96/00394, filed on Feb. 21, 1996.

(30) Foreign Application Priority Data
Feb. 22, 1995 (JP) 033960-1995

(51) Int. Cl. A61K 31/44; A01N 43/40; A61K 31/47; A01N 43/42

(52) U.S. Cl. 514/277; 514/338; 514/310

(57) ABSTRACT

A pharmaceutical composition for treating a transient ischemic attack which comprises a compound of the formula:

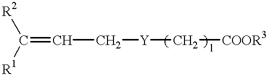

wherein R¹ is a pyridyl group, R² is a phenyl, thienyl, furyl, naphthyl, benzothienyl or pyridyl group, which may be substituted with a lower alkoxy group, a lower alkyl group, a halogen atom, trifluoromethyl group, a lower alkenyl group or/and methylenedioxy group, R³ is hydrogen atom or a lower alkyl group, and l is an integer of 0 to 6, Y is sulfur atom, methylene group or a group of the formula:

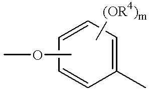

wherein R⁴ is hydrogen atom or acetyl group, and m is 0 or 1, or a pharmaceutically acceptable salt.

### TECHNICAL FIELD

The present invention relates to a pharmaceutical composition for treating or preventing a transient ischemic attack exhibiting therapeutic and prophylactic activities against transient ischemic attack (TIA).

### BACKGROUND ART

TIA, a symptom that precedes cerebral stroke and disappears in short time, is positioned as a prodromal or alerting attack in ischemic cerebral disease. It is generally held that there is a high risk of gradual conversion of TIA to a severe cerebrovascular disorder, such as cerebral infarction, and that the onset and recurrence of severe cerebral disorder can be treated by treating TIA.

By NIH (National Institute of Health) Diagnostic Criteria for TIA Patients (the Classification of Cerebrovascular Diseases, III, Stroke, Vol. 21:653-654, 1990), TIA is an attack characterized by short-term onset of local cerebral dysfunction attributable to ischemia. It is normally confined to a single vascular system (left or right common carotid arterial system, or vertebral vasilar system), involving no other causes. It is common practice to define TIA as an attack that lasts for no longer than 24 hours. The longer the attack, the more often cerebral infarction lesions are detected in computed tomography (CT) or magnetic resonance imaging (MRI). TIA normally lasts for 2 to 15 minutes, its onset being drastic (syndrome reaching peak within 5 minutes, mostly within 2 minutes). Very short attacks, that last for no longer than several seconds, are not viewed as TIA. Although TIA does not leave permanent nerve lesions, there are often relapses. As well, there are atypical cases to which the above definition does not apply.

By the “Diagnostic Criteria for Cerebrovascular Disorders-Classification by Pathologic Form and Severity” (Kameyama et al., Naika, 55(6):1306, 1985), TIA associated with the internal carotid arterial system is characterized by one or more symptoms of motor disorder, loss of vision, sensory disturbance and aphasia. Symptoms of TIA associated with the vertebral vasilar system also include nerve symptoms such as motor disorder, eye symptoms and vertigo, which develop singly or in combination.

On the other hand, EP-B-98690 describes that the compound of the formula (I) below and salts thereof possess thromboxane synthetase-inhibiting activity.

### DISCLOSURE OF INVENTION

With regard to the mechanism of TIA onset, cerebral vasospasm, microembolization, cerebrovascular failure and other factors have been proposed as causative, the latter two being viewed as likely. To prevent and treat TIA, anticoagulation, antiplatelet and other therapies have been attempted, with some effect. Drugs used in such therapies include aspirin and ticlopidine; however, there is demand for a more effective drug that is clinically tolerable, with less severe adverse effects, in chronic administration.

The present inventors sought a compound that treats TIA, and clinically confirmed, for the first time, that a compound known as a thromboxane synthetase inhibitor is effective against TIA. The inventors investigated further based on this finding, and developed the present invention.

The present invention relates to

(1) a pharmaceutical composition for treating a transient ischemic attack (TIA) which comprises a compound of the formula:

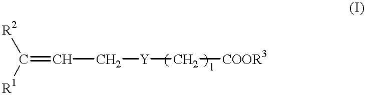

wherein R¹ is a pyridyl group, R² is a phenyl, thienyl, furyl, naphthyl, benzothienyl or pyridyl group, which may be substituted with a lower alkoxy group, a lower alkyl group, a halogen atom, trifluoromethyl group, a lower alkenyl group or/and methylenedioxy group, R³ is hydrogen atom or a lower alkyl group, and l is an integer of 0 to 6, Y is sulfur atom, methylene group or a group of the formula:

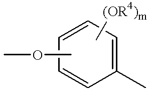

wherein R⁴ is hydrogen atom or acetyl group, and m is 0 or 1, or a pharmaceutically acceptable salt,

(2) a pharmaceutical composition as defined in (1) above, which comprises a compound of the formula:

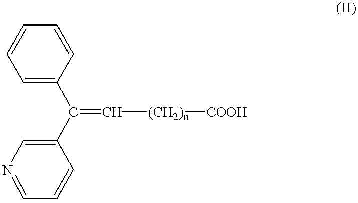

wherein n is an integer of 2 to 6, or a pharmaceutically acceptable salt, and

(3) a pharmaceutical composition as defined in (1) above, wherein the compound is 7-phenyl-7-(3-pyridyl)-6-heptenic acid.

In the above formula (I), R¹ represents a pyridyl group, and R² represents a phenyl, thienyl, furyl, naphthyl, benzothienyl or pyridyl group, which may be substituted with a lower alkoxy group, a lower alkyl group, a halogen atom, trifluoromethyl group, a lower alkenyl group or/and methylenedioxy group.

In the above formula (I), the pyridyl group of R¹ or R² is exemplified by 2-pyridyl, 3-pyridyl and 4-pyridyl, and, the thienyl, furyl, naphthyl and benzothienyl are exemplified by 2-thienyl and 3-thienyl; 2-furyl and 3-furyl; α-naphthyl and β-naphthyl; 2-benzothienyl, 3-benzothienyl, 4-benzothienyl, 5-benzothienyl, 6-benzothienyl and 7-benzothienyl.

As the substituents of said phenyl, furyl, thienyl, naphtyl, benzothienyl and pyridyl of R² which may be substituted, mention is made of lower alkoxy groups (C₁₋₄ alkoxy groups such as methoxy, ethoxy, n-propoxy, i-propoxy, n-butoxy, i-butoxy, t-butoxy, etc.), lower alkyl groups (C₁₋₅ alkyl groups such as methyl, ethyl, n-propyl, i-propyl, n-butyl, s-butyl, i-butyl, t-butyl, n-pentyl, i-pentyl, etc.), halogen atoms (fluorine, chlorine, bromine, iodine, etc.) and lower alkenyl groups (C₂₋₅ alkenyl groups such as vinyl, allyl, pentenyl, etc.). The phenyl, thienyl, furyl, naphthyl, benzothienyl or pyridyl of R² optionally has 1 to 5 of these substituents at any substituable positions of the ring. Preferable example of R¹ includes 3-pyridyl. Preferable example of R² includes phenyl.

In the above formula (I), R³ represents hydrogen atom or a lower alkyl group. As the lower alkyl of R³ in the above formula (I), mention is made of C₁₋₄ alkyl groups of such as methyl, ethyl, n-propyl, i-propyl, n-butyl, i-butyl, s-butyl, t-butyl, etc. Preferable example of R³ includes hydrogen atom.

In the above formula (II), Y represents sulfur atom, methylene group or a group of the formula:

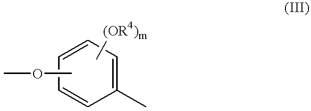

wherein R⁴ is hydrogen atom or acetyl group, and m is 0 or 1. In the above formula (III), R⁴ represents hydrogen atom or acetyl group, and m represents 0 or 1. Preferable example of Y includes methylene group.

In the above formula (I), l represents an integer 0 to 6. Preferable examples of l include an integer of 0 to 4, 2 is more preferably.

Preferable examples of the compound of the formula (I) include a compound, wherein R¹ is 3-pyridyl, R² is phenyl, R³ is hydrogen atom, Y is methylene group and l is an integer of 0 to 4, namely a compound of the formula (II).

In the above formula (II), n represents an integer 2 to 6. Preferable example of the compound of the formula (II) include a compound, wherein n is 4. Example of a representative compound represented by the formula (I) and (II) above includes 7-phenyl-7-(3-pyridyl)-6-heptenic acid.

A compound of the formula (I) above or a pharmacologically acceptable salt thereof can easily be produced by the method described in the above-mentioned patent publication (EP-B-98690).

The pharmacologically acceptable salt of a compound of the formula (I) is exemplified by salts with mineral acids such as hydrochloric acid, sulfuric acid and phosphoric acid, salts with organic acids such as methanesulfonic acid, benzenesulfonic acid, malic acid, citric acid and succinic acid, salts with alkali metals such as sodium and potassium, salts with alkaline earth metals such as calcium and magnesium, and salts with basic amino acids such as arginine. These salts can easily be produced by bringing a compound of the formula (I) into contact with acid or alkali.

The present composition comprising a compound of the formula (I) or a pharmacologically acceptable salt thereof has not significantly toxic to various animal species and very safe to humans. Therefore, the present composition is useful for treating or preventing TIA.

A compound of the formula (I) or a pharmacologically acceptable salt thereof can be prepared as it is or together with pharmaceutically acceptable carriers and/or additives into preparations in dosage form such as tablets, powders, capsules, granules, fine granules, sustained-release preparations or injectable preparations, by known pharmaceutical production techniques, and is normally administered to mammals (e.g. mouse, rat, hamster, rabbit, cat, dog, caw, horse, sheep and monkey), including humans, by oral, subcutaneous, intramuscular, intravenous or other methods. Oral administration is preferred. The present composition comprising a compound of the formula (I) or a pharmacologically acceptable salt thereof is administered normally at 20-200 mg/day, preferably 20-150 mg/day as a compound of the formula (I) or a pharmacologically acceptable salt thereof per adult (weighting 50-70 kg) for oral administration, dividing 1 to 4 times, for treating TIA, depending on symptoms, route of administration etc.

The content of a compound of the formula (I) or a pharmacologically salt thereof in the pharmaceutical preparation of the present invention ranges usually from 0.1 to 100 weight %, preferably from 1 to 50 weight %, relative to the whole weight of the pharmaceutical preparation.

The carriers which the present pharmaceutical preparations include adequately is selected from excipients (e.g. calcium carbonate, kaolin, sodium hydrogencarbonate, lactose, starch (e.g. corn starch), crystalline cellulose (e.g. microcrystallized cellulose), talc, saccharose and porous substance), binders (e.g. dextrin, gum (e.g. arabic gum), alcoholated starch, gelatin, hydroxypropyl cellulose, hydroxypropyl methyl cellulose and pullulane), disintegrants (e.g. carboxymethyl cellulose calcium, closcarmellose sodium, clospovidone, low-substituted hydroxypropyl cellulose and partial a-starch), lubricants (e.g. magnesium stearate, calcium stearate, talc, starch and sodium benzoate), colorants (e.g. tar pigment, caramel, iron sesquioxide, titanium oxide and riboflavins), flavoring agents (e.g. sweeteners and perfume), stabilizers (e.g. sodium sulfite) and preservatives (e.g. parabens and sorbic acid) in adequate amounts respectively. The sustained-release preparations can be produced by coating tablets, granules, fine granules or capsules with, for examples, oil and fat (e.g. triglyceride), ester of fatty acid of polyglycerine or hydroxypropyl cellulose, by per se known means. As the carriers for injectable preparations, use is made of, for examples, distilled water, physiological saline solution, glucose solution, an agent of infusion, sodium chloride and sodium hydroxide.

The present composition comprising a compound of the formula (I) or a pharmacologically acceptable salt thereof can be used, at the same time or at intervals, in combination with antidementic agents, nitrogen monoxide inhibitors, glutamate inhibitors, vascular hypertrophy inhibitors etc., as well as with cerebral circulation and blood flow-improving agents, cerebral metabolism-improving agents, hypertension remedies, diabetes mellitus remedies, anti-cerebral edema agents, thrombolytic agents, lipid metabolism-improving agents and radical scavengers.

In the combination with the present composition, cerebral circulation and blood flow-improving agents include Calan (trade name) (common name: vinpocetine); cerebral metabolism-improving agents including Avan (trade name) (common name: idebenone); hypertension remedies including Adecut (trade name) (common name: delapril hydrochloride), Calslot (trade name) (common name: manidipine hydrochloride) and Candesartan cilexetil; diabetes mellitus remedies including Basen (trade name) (common name: Voglibose) and sulfonyl urea agent; anti-cerebral edema agents including glycerol; thrombolytic agents including tissue plasminogen activator and prourokinase; lipid metabolism-improving agents including Mevalotin (trade name) (common name: pravastatin sodium) and Amotril (trade name) (common name: clofibrate); and radical scavengers including vitamins E and C.

### BEST MODE FOR CARRYING OUT THE INVENTION

### Working Example

The present invention is hereinafter described in more detail by means of the following example.

### EXAMPLE 1

(Tablet)

| | | |
|---|---|---|
| | 50 mg | 100 mg |
| Ingredient | Tablet | Tablet |

| | | | | |
|---|---|---|---|---|
| (E)-7-(3-pyridyl)-7-phenyl-6- | 50.0 | | 100.0 | |
| heptenic acid (test compound) | | | | |
| Lactose | 21.4 | | 42.8 | |
| Corn starch | 15.65 | | 31.3 | |
| Hydroxypropyl cellulose | 2.6 | | 5.2 | |
| Magnesium stearate | 0.35 | | 0.7 | |
| Total | 100.0 | mg | 180.0 | mg |

### EXAMPLE 2

(Sugar coated tablet)

| Ingredient | | |
|---|---|---|
| the above 100 mg Tablet | 180.0 | |
| Talc | 30.0 | |
| Gum arabi | 6.0 | |
| Saccharose | 74.0 | |
| Total | 290.0 | mg |

The tablet obtained in Example 1 was coated to give sugar coated tablet.

### EXAMPLE 3

(Capsule)

| Ingredient | | |
|---|---|---|
| (E)-7-(3-pyridyl)-phenyl-6- | 10.0 | |
| heptenic acid | | |
| Crystallite cellulose | 30.0 | |
| Loctose | 57.0 | |
| Magnesium stearate | 3.0 | |
| Total | 100.0 | mg |

The above components were mixed and the gelatine capsule was filled to capsule.

### EXAMPLE 4

(Injectable preparation)

| Ingredient | |
|---|---|
| (E)-7-(3-pyridyl)-7-phenyl-6- | 2.0 |
| heptenic acid | |
| sodium chloride | 8.45 |
| 1/10 Sodium hydroxide | adequate |
| | amount |
| Water | all amount |
| | 1 ml |
| pH | 8.5-9.0 |

The above components were mixed to give injectable preparation.

### Test

Clinical Effect on TIA

The protocol outline of, and the results from, the phase III clinical study of TIA are shown below.

Study Design

This study was conducted in accordance with Good Clinical Practice (GCP).

Subjects and total number: The subjects of this study were patients who developed one or more TIA attacks associated with the internal carotid arterial system (NIH Diagnostic Criteria, 1990) during the 3-month period before initial administration (171 for efficacy and utility, 175 for safety).

Investigational drug and method of administration:

Tablets each containing 50 mg or 100 mg of the subject compound obtained in Example 1 were orally administered after breakfast once daily for 18 months. The study was conducted in double blind fashion.

Contraindicated concomitant drugs: Concomitant use of ticlopidine, aspirin preparations, heparin, warfarin and ozagrel was prohibited. If in use, they were discontinued.

Items of observation and assessment, and time schedule:

TABLE 5
Evaluation time and evaluation items

| | | After treatment for | | | | | | | | | |
|---|---|---|---|---|---|---|---|---|---|---|---|
| observation, | | two | 1 | 2 | 3 | 6 | 9 | | 1 year and | 1 year and | Finish, |
| evaluation items | Start | weeks | month | months | months | months | months | 1 year | 3 months | 6 months | drop out |
| Patients history and | ● | | | | | | | | | | |
| informed consent | | | | | | | | | | | |
| CT: Computed tomography | ● | | | | | | | | ←●→ | | ● |
| MRI: Magnetic Resonance | | | | | | | | | | | |
| Imaging | | | | | | | | | | | |
| Carebral angiography | ∘ | | | | | | | | | | ● |
| Incidence of TIA and clinical | ←●→ | | | | ● | ● | ● | ● | ● | ● | ● |
| manifestation | 3 months | | | | | | | | | | |
| Electrocardiography | ● | | | | | | | | | ● | ● |
| Side effects, complications | | ← any time → | | | | | | | | | ● |
| Clinical tests | ● | ●* | ●* | ●* | ● | ● | ●* | ● | ●* | ● | ● |
| Evaluation of effectiveness | | | | | | ● | | ● | | ● | ● |

●: essential
●*: blood chemistry and liver function test
∘: if at all possible

Notes

2) CT (or MRI) examination

CT (or MRI) was conducted before initial administration and at 15 months to 18 months of administration. CT (or MRI) examination was conducted before and after administration, in as similar a fashion as possible.

3) Cerebral angiography

Whenever possible, cerebral angiography was performed to assess the affected blood vessel before study initiation.

4) ECG

ECG was performed before initial administration and at 18 months of administration.

5) Clinical signs of TIA

TIA onset and the following items were examined at least every 3 months during the 3 months before initial administration and during the administration period.

| | | |
|---|---|---|
| \mathring{1} | Date of onset | |
| \mathring{2} | Diagnosis: | TIA (internal carotid arterial |
| | | system, vertebral vasilar system), |
| \mathring{3} | Duration | |
| \mathring{4} | Syndromes: | Sensory disturbance (numbness, |
| | | anesthesia), motor disorder (site), |
| | | vision disorder (description), |
| | | speech disorder (aphagia, alalia), |
| | | others |
| \mathring{5} | Condition: | Diurnal activity, just after |
| | at attack | wakening, at rest, at sleep, others |

6) Side effects, complications (cerebral, cardiac, peripheral vascular disorder onset and complications)

If any of the following cerebral, cardiac and peripheral vascular disorders occurs (including complications) during the study period, its diagnosis, syndrome, course of disease, treatment etc. were recorded.

| \mathring{1} | Cerebral vascular: | Cerebral infarction |
|---|---|---|
| | disorder | (severity: mild, moderate |
| | | and higher) |
| Note: | | |
| “Mild” was defined as a state of disease in which the | | |
| syndrome lasts for not less than 24 hours and not more than | | |
| 3 weeks. | | |
| Cerebral hemorrhage, subarachnoid hemorrhage | | |
| \mathring{2} | Cardiac diseases: | Myocardial infarction, |
| | | angina pectoris |

| | |
|---|---|
| \mathring{3} | Peripheral and retinal arterial thrombotic diseases etc. |

7) Clinical laboratory testing

The following parameters were tested at initial administration and 3 months, 6 months, 1 year and 18 months of administration.

Hematology and liver function also were tested at 2 weeks, 1 month, 2 months, 9 months and 15 months of administration.

| | |
|---|---|
| Physical | Blood pressure, pulse rate |
| examination | |
| Hematology | Red blood cell count, hemoglobin |
| | content, hematocrit value, white |
| | blood cell count, platelet count, |
| | differential white blood cell count |
| Blood bio- | (Liver function) |
| chemistry | Total protein, GOT, GPT, γ-GTP, |
| | Al-P, LDH |
| | CK, total cholesterol, HDL-cholesterol, |
| | triglyceride (at hunger), BUN, |
| | creatinine, fasted blood glucose |
| Urinalysis | Protein, sugar, urobilinogen, occult |
| | blood |

If an abnormal change (worsened from pre-administration value) is noted during the therapeutic period, follow-up survey was performed to determine relation to the test drug.

8) Overall assessment

(1) Efficacy in suppressing TIA recurrence

As regards conversion to cerebral infarction, temporal changes in TIA onset frequency, TIA symptoms and duration, etc., efficacy was rated at 6 months, 1 year and 18 months of administration, using a 5-grade rating system:

1. Effective

2. Slightly effective

3. Ineffective

4. Worsened

5. Indeterminable

(Judgment criteria)

The attack frequency at time of rating (during therapeutic period) 1 year and 18 months after initial administration served as an efficacy rating index. For rating at 6 months of administration and rating at less than 1 year due to discontinuation (adverse reactions etc.), rating was in regard to duration of administration period, with reference to the following rating criteria.

TABLE 6
Rating Criteria for Efficacy in Suppressing TIA
Recurrence (at 1 year and 18 months of administration)

| Number of Attacks | | | | |
|---|---|---|---|---|
| during 3 Months | | | | |
| before | Number of Attacks during the Study Period | | | |
| Administration | 0 | 1 | 2 | 3 or More |
| 1 | Effective | Ineffective | Ineffective | |
| | or | | or worsened | |
| | slightly | | | |
| | effective | | | |
| 2 | Effective | Slightly | Ineffective | |
| | | effective | or worsened | |
| 3 | Effective | Effective | Slightly | Ineffective |
| | | | effective | or worsened |
| 4 or more | Effective | Effective | Effective | *) |

Duration of administration period was considered when rating at 6 months of administration and at less than 1 year due to discontinuation (adverse reactions etc.).
Note 1)
If a cerebral infarction attack is noted during the study period, the rating shall be “worsened.” If myocardial infarction or peripheral and retinal arterial thrombotic disease is noted, the rating shall be “ineffective” or “worsened,”.
Note 2)
If administration is discontinued due to onset of TIA attack during the study period, the rating shall be “ineffective.”
*)Not more than 50% of the number of pre-administration attacks: Effective
75-50% of the number of pre-administration attacks: Slightly effective
More than 75% of the number of pre-administration attacks: Ineffective or worsened

(2) Overall safety

Overall safety was rated on the basis of adverse reactions, complications, and presence or absence of abnormal changes in clinical laboratory parameters and their degrees, using a 5-grade rating system:

1. No problem

2. Slightly problematic

3. Considerably problematic

4. Very problematic

5. Indeterminable

(3) Utility

On the basis of efficacy and overall safety ratings for suppression of TIA recurrence, utility was rated using a 5-grade rating system:

1. Useful

2. Slightly useful

3. Not useful

4. Undesirable

5. Indeterminable

However, if the overall safety rating was “considerably problematic” or “very problematic,” the utility rating “useful.”

Data Analysis

With regard to the above rating items, data were analyzed using ITT (intent-to-treat analysis), with the utility rating in overall assessment at final administration (18 months after administration) as the major item.

Results

The results of efficacy, overall safety and utility ratings of the test compound are shown in Tables 7 through 9.

The utility rates of the test compound in the 50 mg and 100 mg dose groups were 60.0 and 60.5%, respectively (Table 9). The efficacy rates of the test compound at 50 mg and 100 mg were 64.7 and 66.3%, respectively (Table 7). The overall safety rates of the test compound at 50 and 100 mg were 86.0 and 79.8%, respectively (Table 8). In conclusion, the test compound is a useful drug for TIA patients.

TABLE 7
Efficacy (%)

| | | Slightly | | | | Number |
|---|---|---|---|---|---|---|
| Drug | Effec- | Effec- | Inef- | | Inde- | of |
| (dose/day) | tive | tive | fective | Worsened | terminable | Patients |
| Test | 64.7 | 2.3 | 16.5 | 5.9 | 10.6 | 85 |
| compound | | | | | | |
| (50 mg) | | | | | | |
| Test | 66.3 | 7.0 | 8.1 | 7.0 | 11.6 | 86 |
| compound | | | | | | |
| (100 mg) | | | | | | |

TABLE 8
Overall Safety (%)

| | | | Con- | | | |
|---|---|---|---|---|---|---|
| | | Slightly | siderably | Very | | Number |
| Drug | No | Prob- | Prob- | Prob- | Inde- | of |
| (dose/day) | Problem | lematic | lematic | lematic | terminable | Patients |
| Test | 86.0 | 8.1 | 1.2 | 0 | 4.7 | 86 |
| compound | | | | | | |
| (50 mg) | | | | | | |
| Test | 79.8 | 13.5 | 1.1 | 2.2 | 3.4 | 89 |
| compound | | | | | | |
| (100 mg) | | | | | | |

TABLE 9
Utility (%)

| | | | | | | Number |
|---|---|---|---|---|---|---|
| Drug | | Slightly | Not | Un- | Indeter- | of |
| (dose/day) | Useful | Useful | Useful | desirable | minable | Patients |
| Test | 60.0 | 5.9 | 23.5 | 5.9 | 4.7 | 85 |
| compound | | | | | | |
| (50 mg) | | | | | | |
| Test | 60.5 | 10.5 | 16.3 | 9.3 | 3.5 | 86 |
| compound | | | | | | |
| (100 mg) | | | | | | |

### Industrial Applicability

A compound of the formula (I) or a pharmacologically acceptable salt thereof treats or prevents transient ischemic attack.

1. A pharmaceutical composition for treating a transient ischemic attack which comprises a compound of the formula: 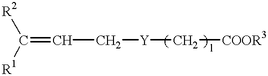
wherein R¹ is a pyridyl group, R² is a phenyl, thienyl, furyl, naphthyl, benzothienyl or pyridyl group, which may be substituted with a lower alkoxy group, a lower alkyl group, a halogen atom, trifluoromethyl group, a lower alkenyl group or/and methylenedioxy group, R³ is hydrogen atom or a lower alkyl group, and l is an integer of 0 to 6, Y is sulfur atom, methylene group or a group of the formula: 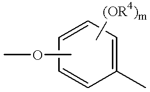
wherein R⁴ is hydrogen atom or acetyl group, and m is 0 or 1, or a pharmaceutically acceptable salt.

2. A pharmaceutical composition according to claim 1, which comprises a compound of the formula: 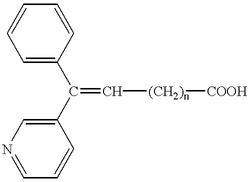
wherein n is an integer of 2 to 6, or a pharmaceutically acceptable salt.

3. A pharmaceutical composition according to claim 1, wherein the compound is 7-phenyl-7-(3-pyridyl)-6-heptenic acid.

4. A method of treating a transient ischemic attack in a mammal which comprises administering to the mammal an effective amount of a compound of the formula: 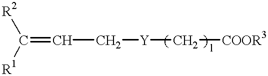
wherein R¹ is a pyridyl group, R² is a phenyl, thienyl, furyl, naphthyl, benzothienyl or pyridyl group, which may be substituted with a lower alkoxy group, a lower alkyl group, a halogen atom, trifluoromethyl group, a lower alkenyl group or/and methylenedioxy group, R³ is hydrogen atom or a lower alkyl group, and l is an integer of 0 to 6, Y is sulfur atom, methylene group or a group of the formula: 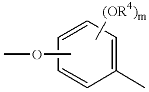
wherein R⁴ is hydrogen atom or acetyl group, and m is 0 or 1, or a pharmaceutically acceptable salt.

5. Use of a compound of the formula: 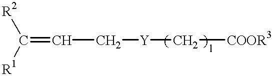
wherein R¹ is a pyridyl group, R² is a phenyl, thienyl, furyl, naphthyl, benzothienyl or pyridyl group, which may be substituted with a lower alkoxy group, a lower alkyl group, a halogen atom, trifluoromethyl group, a lower alkenyl group or/and methylenedioxy group, R³ is hydrogen atom or a lower alkyl group, and l is an integer of 0 to 6, Y is sulfur atom, methylene group or a group of the formula: 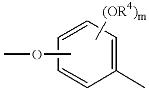
wherein R⁴ is hydrogen atom or acetyl group, and m is 0 or 1, or a pharmaceutically acceptable salt, for preparing a pharmaceutical composition for treating a transient ischemic attack.
